(19) United States
(12) Patent Application Publication
(10) Pub. No.: US 2003/0215387 A1
(43) Pub. Date: Nov. 20, 2003

(54) Bifunctional antibodies and their use in targeting anti-tumour agents

(76) Inventor: Peter Harrison, Surrey (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/445,132

(22) Filed: May 22, 2003

Related U.S. Application Data
(63) Continuation of application No. 09/786,013, filed on Apr. 24, 2001, now abandoned.
Application No. 09/786,013 is a 371 of international application No. PCT/GB99/02938, filed on Sep. 6, 1999.

(30) Foreign Application Priority Data
Sep. 4, 1998 (GB) 9819411.1

(51) Int. Cl. A61K 51/00; C07K 16/46

(52) U.S. Cl. 424/1.49; 530/391.1

(57) ABSTRACT

A bifunctional antibody has affinity for a target site and affinity for an organic molecule covalently linked to a cytotoxic agent or an enzyme capable of converting a prodrug into its cytotoxic form. The antibody may be used in therapy or diagnosis, especially in the treatment of tumours.

### FIELD OF THE INVENTION

This invention relates to bifunctional antibodies and to their use in targeting anti-tumour agents in vivo.

### BACKGROUND TO THE INVENTION

The targeting of therapeutic agents to particular sites in vivo, is well known. In particular, it is very desirable to target anti-cancer agents to a tumour site, to increase the concentration of the agent at the site and thereby improve its effectiveness in neutralising the tumour. Examples of agents that target tumours are well known, many of these relying on the specificity of monoclonal antibodies for delivering the diagnostic or therapeutic agent to the target site. One approach has been to use a radionuclide-antibody conjugate which localises at a target tissue where the radionuclide may exert its cytotoxic effect.

However, it has been shown that there are problems with the utility of radionuclide-antibody conjugates, for example poor penetration of the conjugates to the target site owing to the high molecular weight of the conjugate. In addition, for the conjugate to be therapeutically effective, it must be given time to localise at the target site. The radionuclide is therefore present in the body for prolonged periods, and this results in undesirable toxicity at non-target sites.

It is therefore desirable to administer the antibodies independently of the cytotoxic radiolabelled agent, allowing the antibody to localise at the target site before administering the cytotoxic radiolabelled agent.

U.S. Pat. No. 5,630,996 describes one approach that uses antibody-streptavidin conjugates to target a radionuclide-labelled biotin. Streptavidin has high affinity for biotin and is able to localise the radionuclide at the target site through the biotin-streptavidin interaction. However, streptavidin is a protein that is immunogenic in humans and consequently may not be suitable for repeated long-term therapeutic use.

U.S. Pat. No. 5,591,828 discloses bispecific antibodies that have affinity for metal chelates and for a particular protein epitope. However, the chelates, EDTA-Y⁹⁰ and DTPA-Y⁹⁰ preferably require the covalent addition of a fos-peptide which interacts with a jun-peptide on the antibody, to localise sufficient Y⁹⁰ to the target site. In addition, dissociation may occur between the chelating agent (EDTA) and the radionuclide (Y⁹⁰). Therefore, localisation at the tumour site may not be efficient, and the process of producing the fos-peptide-metal chelate conjugate is lengthy and unsuitable for large-scale manufacture.

### SUMMARY OF THE INVENTION

The present invention relates to bifunctional antibodies which have affinity for both an antigen present at a tumour site, and for a therapeutic or diagnostic agent, wherein the agent is an organic molecule that is radiolabelled or is covalently bound to a cytotoxic agent. In a separate embodiment, the organic molecule is bound to an enzyme capable of converting a prodrug into a cytotoxic form.

The invention therefore provides a means for increasing the localisation of a therapeutic agent at a target site in a mammal, by the sequential administration of the bifunctional antibody and the organic molecule. The invention may have use in therapeutic or diagnostic applications.

Typically, the organic molecule is biotin or an organic molecule which exhibits good penetration at a target site and otherwise neutral biodistribution in vivo.

One of the advantages of the present invention, is that it is possible to administer a cytotoxic agent which will localise at the desired target due to the interaction with antibody, but which will have reduced toxicity to non-target tissues, as unbound agent will be cleared efficiently from the body.

In one embodiment, the cytotoxic agent is a radionuclide, and is covalently linked to the organic molecule, or is itself part of the organic molecule. The radionuclide may be chosen to provide a therapeutic effect, e.g. as an anti-tumour agent, or may be administered for diagnostic purposes, e.g. tumour imaging.

In another embodiment, the organic molecule is linked to an enzyme that can be used to convert a suitable prodrug into an active cytotoxic form.

### DESCRIPTION OF THE INVENTION

The antibodies of the present invention may be produced using conventional techniques, for example, hybridoma synthesis, recombinant DNA techniques or phage display. The antibodies may be derived from any species, including rodent, although it is preferred that the antibodies are derived from mammals other than rodents, e.g. sheep, goats or cows, to generate high-affinity antibodies.

Typically, the antibodies will have an affinity of at least 10¹⁰ l/mol, preferably 10¹¹ l/mol, more preferably 10¹² l/mol and most preferably 10¹³ l/mol for the respective ligands.

A bifunctional antibody according to the invention may be whole antibody or may be a fragment thereof, e.g. f(ab)₂. In a further embodiment, the antibody may comprise two single chain fv fragments. The preparation of bifunctional sFvs is well known. For example, Carter et al., Current Opinion in Biotechnology 1997, 8: 449-454, discloses the production of bifunctional sFvs using phage display libraries.

In addition, the antibodies may be modified by recombinant DNA techniques to “humanise” the antibodies, making them less immunogenic when administered to a patient. The humanised antibody should comprise at least the hypervariable region from both a monoclonal antibody having affinity for target antigen, and a monoclonal antibody having affinity for the organic molecule. The remainder of the antibody variable region may be of human immunoglobulin. A higher proportion of human immunoglobulin may be present in a whole antibody or a fragment, e.g. F(ab′)₂ When a single-chain Fv fragment is used, the fragment may comprise hypervariable regions as described above and, optionally, the variable framework from human immunoglobulin.

The antibody will have affinity for a particular target site. Typically, the target site will be a tumour and the antibody will have affinity for a tumour-associated antigen. An example of a tumour-associated antigen is the carcinoembryonic antigen (CEA) which is found on colorectal tumours and other adeno-carcinomas.

In the preferred embodiment, the antibody has ligand-affinity for an organic molecule that is radiolabelled. The invention encompasses both the separate covalent attachment of a radionuclide to an organic molecule, and additionally the simple radiolabelling of a suitable atom on the organic molecule itself. For example, the organic molecule may comprise a phosphorous or iodine atom which is radiolabelled to provide a cytotoxic organic molecule. Administration of the molecule will localise the radionuclide at a tumour site through binding to the antibody to exert a cytotoxic effect on the tumour. Radionuclides having a cytotoxic effect are well known. A preferred radionuclide that may be used in the invention is a radioisotope of iodine, e.g. I¹²³, I¹²⁴ and I¹²⁵ which may be used for diagnostic purposes and I¹³¹ which may be used in therapeutics. A further preferred radionuclide that may be used in the invention, is P³²,

The cytotoxic agent may also be a cytotoxic drug, e.g. ricin or calicheamycin.

In an alternative embodiment, the organic molecule is linked (conjugated) to an enzyme. The enzyme is capable of converting a suitable prodrug into an active cytotoxic form. The term “prodrug” is used herein to define an inactive form of a drug which may be cleaved by enzymic action to release the therapeutically-active form. Suitable enzyme-prodrug systems are known to those skilled in the art and include carboxypeptidases and modified mustard gas derivatives.

The organic molecules that are useful in the present invention must be capable of specific interaction with an antibody. The molecules must therefore be of a sufficient size to elicit an immune response for the production of the antibodies when conjugated to a protein carrier, or be of a sufficient size to facilitate antibody creation from antibody libraries, e.g. displayed on filamentous phage. Preferably, the organic molecules linked to the cytotoxic agent or enzyme are capable of passing through the lining of the vasculature to reach the target site. The molecular weight of the organic molecules is preferably less than 1500, more preferably less than 1000. The organic molecules are preferably non-toxic when not radiolabelled. In addition, the labelled organic molecules should have a neutral biodistribution when administered to a patient in the absence of bispecific antibody, and it is preferable if molecules are chosen which do not accumulate in the thyroid and are rapidly cleared through the kidney. It is also beneficial if the molecules are easily derived from a non-labelled parent molecule and that the derived, labelled molecule is stable following administration. The conjugate should preferably be water-soluble, to facilitate preparation in suitable excipients. Suitable organic molecules include non-toxic compounds, although the molecules may be made cytotoxic by radiolabelling.

In a preferred embodiment, the radio-labelled organic molecule is radio-labelled biotin. In a further preferred embodiment, the molecule is of formula I

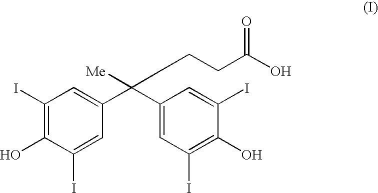

i.e. 4,4-bis(4-hydroxy-3,5-diiodophenyl)pentanoic acid.

In a further preferred embodiment, the molecule is of formula II

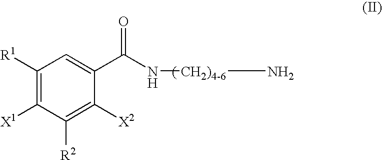

where R¹ and R² are each, independently, a radiolabelled moiety, e.g. radiolabelled iodine, a methyl group or a phenyl group, and X¹ and X² are each, independently, a H or OH.

Preferred radiolabelled molecules are N-(4-hydroxy-3,5-diiodobenzoyl)-1,6-hexanediamine and N-(2-hydroxy-3,5-diiodobenzoyl)-1,6-hexanediamine, where either or each of the iodine atoms may be any of the radioisotopes I¹²³, I¹²⁵, I¹²⁴ and I¹³¹.

For use in the invention, the bispecific antibody and the cytotoxic agent may be formulated in a kit, e.g. comprising the two components separately packaged or in separate containers. Each component may be formulated with a suitable carrier or excipient, examples of which are well known, depending on the route of administration, e.g. oral or intravenous.

The two components will usually be administered sequentially. The effective amount of each may readily be determined by the skilled person, and will depend on typical factors such as the location, severity and spread of the tumour, the condition of the subject etc. It is of course a feature of this invention that the amount of cytotoxic agent that is required will be less than in the absence of the antibody.

I claim:

1. A bifunctional antibody having affinity for a tumour and for a therapeutic or diagnostic agent, wherein the agent is an organic molecule.

2. The bifunctional antibody, according to claim 1, wherein the agent is covalently-bound to an enzyme.

3. The antibody according to claim 1, wherein the ligand-affinity is at least 10¹⁰ l/mol.

4. The antibody according to claim 1, wherein the agent has a molecular weight of less than 1500.

5. The antibody according to claim 1, wherein the agent is water-soluble and has a neutral biodistribution in vivo.

6. The antibody according to claim 1, wherein the agent is radio-labeled and the radio-label is I¹²³, I¹²⁴, I¹²⁵, P³² or I¹³¹.

7. The antibody according to claim 1, wherein the agent is radiolabelled 4,4-bis(4-hydroxy-3,5-diiodophenyl) pentanoic acid.

8. The antibody according to claim 1, wherein the organic molecule has the formula 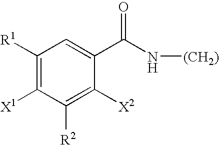
where R¹ and R² are, independently a radiolabel, a methyl group or a phenyl group, and X¹ and X² are, independently, H or OH.

9. The antibody according to claim 8, wherein the agent is radiolabelled N-(4-hydroxy-3,5-diiodobenzoyl)-1,6-hexanediamine or N-(2-hydroxy-3,5-diiodobenzoyl)-1,6-hexanediamine.

10. The antibody according to claim 1, wherein the agent is biotin.

11. The antibody according to claim 1, wherein the antibody affinity for the tumour is via a tumour-associated antigen.

12. The antibody according to claim 11, wherein the antigen is carcinoembryonic antigen.

13. The antibody according to claim 1, wherein the antibody comprises two single chain Fvs.

14. The antibody according to claim 1, wherein the antibody comprises at least the constant regions derived from human immunoglobulin.

15. A product comprising a bifunctional antibody having affinity for a tumour and for a therapeutic or diagnostic agent which is an organic molecule covalently bound to a cytotoxic agent or which is a radiolabelled organic molecule.

16. A method for treatment or diagnosis of a pathological condition wherein said method comprises the administration, to a person or animal in need of said treatment or diagnosis, of an effective amount of radiolabelled 4,4-bis(4-hydroxy-3,5-diiodophenyl)pentanoic acid, radiolabelled N-(4-hydroxy-3,5-diiodobenzoyl)-1,6-hexanediamine.

17. A pharmaceutical or diagnostic composition comprising a bifunctional antibody having affinity for a tumour and for a therapeutic or diagnostic agent, wherein the agent is an organic molecule.

18. The composition, according to claim 17, wherein said composition is a pharmaceutical composition.

19. The composition, according to claim 17, wherein said composition is a diagnostic composition.

20. A method for treating cancer wherein said method comprises administering to a human or animal in need of such treatment an effective amount of a bifunctional antibody having affinity for a tumour and for a therapeutic or diagnostic agent, wherein the agent is an organic molecule.

21. A diagnostic procedure wherein said procedure utilizes a bifunctional antibody having affinity for a tumour and for a therapeutic or diagnostic agent, wherein the agent is an organic molecule to detect a condition.
